(19) United States
(12) Patent Application Publication
(10) Pub. No.: US 2009/0198129 A1
(43) Pub. Date: Aug. 6, 2009

(54) CHARACTERIZATION OF VULNERABLE PLAQUE USING DYNAMIC ANALYSIS

(76) Inventors: Tomy Varghese, Madison, WI (US); Robert J. Dempsey, Madison, WI (US); Bruce P. Hermann, Madison, WI (US)

Correspondence Address: WISCONSIN ALUMNI RESEARCH FOUNDATION, C/O BOYLE FREDRICKSON S.C, 840 North Plankinton Avenue, Milwaukee, WI, 53203, US

(21) Appl. No.: 12/026,365

(22) Filed: Feb. 5, 2008

(51) Int. Cl. A61B 8/00

(52) U.S. Cl. 600/438; 128/898

(57) ABSTRACT

Arterial plaques are evaluated by determining their deformation under the periodic pulsatile force of blood flow. A relationship between plaque deformation and rupture risk is established by measurement of a relationship between deformation and cognitive decline in a sample population. The measured parameters include the maximum accumulated axial strain, maximum lateral displacement and maximum shear strains in soft vulnerable plaques.

### STATEMENT REGARDING FEDERALLY SPONSORED RESEARCH OR DEVELOPMENT

This invention was made with United States government support awarded by the following agencies:
NIH EB003853.

The United States has certain rights in this invention.

### CROSS-REFERENCE TO RELATED APPLICATIONS

### BACKGROUND OF THE INVENTION

Stroke is the leading cause of mortality and the leading cause of disability in the United States.

Stroke can result from blood flow disruptions to the brain caused by plaques and clots forming on the inner walls of the blood vessels and blocking blood flow through the vessels (thrombotic stroke). Alternatively, the obstruction of blood flow can occur when particles or debris in the bloodstream from another location lodges in a smaller vessel (embolic stroke). One source of this debris is ruptured atherosclerotic plaques that otherwise do not present an immediate risk of arterial blockage. Plaques that are prone to rupture are termed “vulnerable plaques”.

Whether a plaque is vulnerable appears to depend on the internal structure of the plaque. Generally, as a plaque forms in the artery, a calcified layer forms over the softer fatty core. Vulnerable plaques have a thin fibrous cap over the top of a soft lipid pool underlying the cap. Large forces of pulsatile blood flow, for example during strenuous exercise, can break this fibrous cap. Plaques having a thick fibrous cap are less likely to rupture.

Current treatment of arterial plaque focuses on the percent blockage (stenosis) of the carotid artery. When blockage reaches a certain amount, a surgical procedure may be undertaken to remove the plaque from the artery or to widen the artery using a stent.

### BRIEF SUMMARY OF THE INVENTION

The present invention attempts to better identify asymptomatic plaques that are prone to rupture releasing clinical emboli into the cerebral blood stream The invention involved two steps. First, the inventors determined that some otherwise asymptomatic plaques nevertheless appeared to be associated with measurable cognitive decline. The inventors believe that these asymptomatic plaques are releasing subclinical emboli. These subclinical emboli are a concern in themselves but also appear to indicate that the plaques are vulnerable to rupture.

Second, the inventors, have determined that these vulnerable, asymptomatic plaques can be distinguished from other asymptomatic plaques earlier and ideally before there is significant cognitive decline, by measurement of the elasticity and mobility of the plaque under the pulsatile force of blood. Plaques that undergo large deformations and thus incur large axial displacements and strains, large lateral displacements and strains and increased shear strains are particularly of interest.

Specifically, the present invention provides apparatus for the characterization of arterial plaque using an imaging system producing images distinguishing plaque and at least a portion of a supporting arterial wall. An electronic computer executes a stored program and receives the acquired images to: (1) isolate movement of the plaque from movement of the supporting arterial wall under a periodic force of pulsatile blood flow; (2) analyze the movement of the plaque to characterize a risk of the plaque rupturing to produce dangerous embolisms; and (3) output an indication of the risk.

It is thus an object of one embodiment of the invention to provide a noninvasive assessment of the vulnerability of plaques, independent of a measurement of stenosis of the blood vessel.

The analysis may determine one or more of: axial strain or displacement in the plaque, lateral strain or displacement of the plaque, and shear in the plaque.

It is thus another object of one embodiment of the invention to provide a set of different measurements that may quantitatively characterize the plaque using standard image data. It is an object of a least one embodiment of the invention to further provide a functionally continuous measurement that allows more sophisticated assessment of risk.

The imaging system may be an ultrasonic imaging system.

It is thus another object of one embodiment of the invention to provide an assessment suitable for use with readily available ultrasonic imaging systems.

The apparatus may isolate movement of the plaque by subtracting out a movement of the supporting arterial wall.

Thus it is an object of one embodiment of the invention to provide a system that may make use of the periodically varying force of blood flow to characterize the plaque in vivo by isolating motion of the plaque from motion of the vessels supporting the plaque.

The apparatus may isolate movement of the plaque by determining movement of the plaque with respect to other portions of the plaque.

It is thus another object of one embodiment of the invention to provide some measurements that are self-referential and thus tend to decrease the effect of movement of the frame of reference.

The electronic computer may provide a display of the images and a cursor for identifying a region of plaque and a region of supporting arterial wall.

It is thus an object of one embodiment of the invention to make use of the expertise of a physician or other healthcare worker to identify the plaque and the arterial wall for analysis.

The apparatus may include a pulse monitoring system, such as an ECG, providing a timing reference to the computer for the analysis.

It is thus another object of one embodiment of the invention to permit synchronization of the measurements of plaque with the timing of arterial blood flow for advanced statistical processing.

When the imaging system is an ultrasound imaging system, the electronic computer may further analyze scatterer size in characterizing the risk of a plaque rupturing.

It is thus another object of one embodiment of the invention to permit additional ultrasonic measurements to be used to characterize and distinguish between vulnerable and stable plaques.

These particular features and advantages may apply to only some embodiments falling within the claims and thus do not define the scope of the invention.

### BRIEF DESCRIPTION OF THE DRAWINGS

FIG. 1 is a block diagram of an ultrasound machine suitable for use with the present invention such as may execute a program for characterizing plaques according to the present invention;

FIG. 2 is a screen display of the ultrasound machine of FIG. 1, showing the placement of region of interest cursors on a “B-mode” or Doppler ultrasound image of a blood vessel and plaque;

FIG. 3 is a figure similar to that of FIG. 2 showing a subsequent image frame and movement of the blood vessel and plaque;

FIG. 4 is a schematic representation of the plaque of FIGS. 2 and 3 showing three different dynamic measurements as may be used in the present invention;

FIG. 5 is a flow chart showing the steps of the program executed by the ultrasound machine of FIG. 1 in providing a risk assessment of plaque; and

FIG. 6 is a regression of cognitive decline versus axial strain representing early data obtained by the present inventors showing the clinical significance of these measurements.

### DETAILED DESCRIPTION OF THE INVENTION

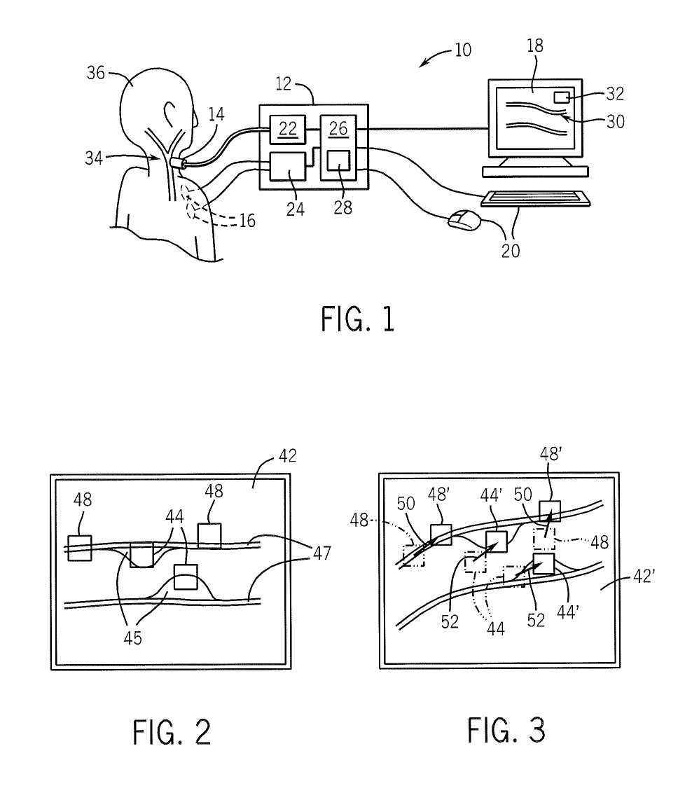

Referring now to FIG. 1, a first embodiment of the present invention may make use of an ultrasound machine 10 having processing unit 12 receiving ultrasonic image data from an ultrasonic transducer 14. The processing unit 12 may further optionally receive cardiac data via one or more ECG electrodes 16 or other pulse monitoring sensors. The processing unit 12 may connect to a display screen 18 and to input devices 20 such as a keyboard, mouse, or other cursor control device for the input of data by an operator.

The ultrasonic transducer 14 may provide radio frequency ultrasonic data to an RF signal processor 22 within the processing unit 12. The RF signal processor 22 provides filtering, envelope extraction (for B-mode imaging), frequency demodulation (for Doppler shift imaging), and other processing techniques well-known in the art of ultrasonic imaging. The ECG electrodes 16, in turn, provide cardiac signals to ECG interface circuitry 24 in the processing unit 12 which may extract cardiac cycle timing information. The RF signal processor 22 and ECG interface circuitry 24, in turn, may communicate with a processor 26 having a stored program 28 implementing standard image formation algorithms to provide an output image 30 on the display screen 18. The processor 26 may also include the program 28 of the present invention, as will be described, to output quantitative risk data 32 on the display screen 18.

The ultrasound signal processing unit 12, for example, may be a Siemens Antares ultrasound system providing an ultrasound research interface package that provides access to radio frequency ultrasonics data. In this case, the processor 26 may be implemented with a freestanding computer providing off-line processing. The ultrasonic transducer 14 may be, for example, a Siemens VFX 13-5 multi-D linear array transducer, or a VFX 9-4 linear array transducer.

Referring still to FIG. 1, the ultrasonic transducer 14 may be directed to the carotid artery 34 of a patient 36 near the branching or bifurcation of the carotid artery 34 and may provide a generally planar beam providing a cross-sectional view of output image 30 of the carotid artery 34 along an axis of the artery.

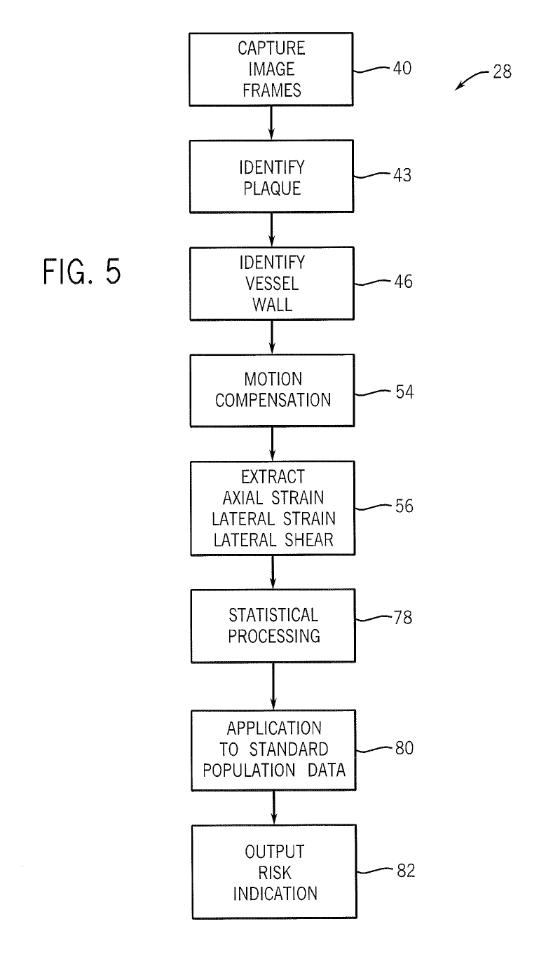

Referring momentarily to FIG. 5, at a first step executed by the program 28, a number of successive image frames, for example, at a rate of approximately 27 frames per second are captured. The image frames provide ultrasonic data including both underlying radiofrequency data and B-mode envelope data and or Doppler ultrasound data, the latter providing an indication of blood flow thus simplifying the identification of arterial plaque.

Referring now also to FIG. 2, a first frame 42 of the captured data may be displayed on the display screen 18 and the physician may position a cursor 44, in this case being an open square, centered on plaque 45 to be evaluated. This process identifies the ultrasound data associated with plaque 45 per process block 43 of FIG. 5.

At succeeding process block 46, one or more additional cursors 48 may be placed on the vessel walls 47, or tissue closely coupled thereto, to provide a reference frame for motion correction.

The program 28 per process block 54 may then process additional image frames 42 and may automatically reposition the cursors 44 and 48 to follow the plaque 45 and vessel wall 47, respectively. This tracking of motion of the tissue may be done by means of a local correlation between the data circumscribed by the cursors 44 and 48 in each given image frame 42 with the data in the next image frame 42′ to establish any movement of the circumscribed material. The cursors 44 and 48 are then repositioned in the next image frame 42′ to the point of highest correlation. By this process, a trajectory of the tissue motion through each image frame 42 may be extracted.

Alternatively, the cursors 44 and 48 may be manually repositioned in each frame 42′ by the physician. The automatic or manual positioning of the cursors 44 and 48 may be aided by Doppler velocity data that may readily distinguish blood flow from occluding material, and that may highlight the point of occlusion by the resulting local high blood velocity

Referring now to FIG. 3, in a succeeding frame 42′, the identified regions of the cursors 48 and 44 of the previous frame 42 may move to new locations indicated by cursors 48′ and 44′. These measured movements 50, between the cursors 48 and 48′ placed on the vessel wall 47 may be used to compensate the movement 52 between cursors 44 and 44′ to isolate movement of the plaque 45 from movement of the underlying tissue. This compensation may be done most simply by subtracting the movements 50 (or an average of those movements 50 when multiple points are measured) from the movements 52. Alternatively, movements 52 may be fit to a compressible model and used to derive interpolated movements 52′ at the location of the plaque 45 to provide motion compensated movement 52.

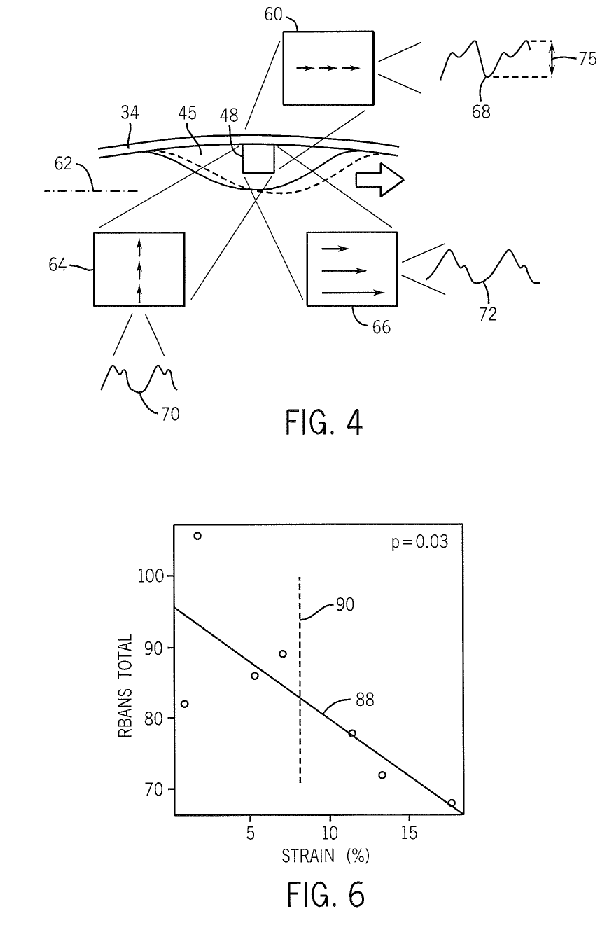

Referring now to FIGS. 4 and 5, at process block 56 of FIG. 5, the motion compensated data underlying the plaque 45 is then analyzed with respect to three dynamic properties deduced by movement of the plaque 45 under the regular periodic force of blood flow. The first of these properties is lateral strain 60, being a change in amount of lateral displacement of elements of the plaque 45 between successive image frames 42, 42′ in a direction along the lumen axis 62. The second of these properties is axial strain 64, being a change in amount of axial displacement of elements of the plaque 45 between successive image data frames 42, 42′, perpendicular to the lumen axis 62. The third of these properties is shear strain 66, being a change in the amount of lateral and axial displacement of elements of the plaque 45 between successive image data frames 42, 42′ as one moves perpendicular to the lumen axis 62.

Techniques for determining strain from the ultrasonic data are described in U.S. Pat. No. 7,166,075, entitled: “Elastographic imaging of in vivo soft tissue”; U.S. Pat. No. 6,749,571, entitled: “Method and apparatus for cardiac elastography” and U.S. patent applications: 2007/0083113, entitled: “High resolution elastography using two step strain estimation”; 2005/0165309, entitled: “Ultrasonic elastography providing axial, orthogonal, and shear strain”; 2004/0243001, entitled: “Parametric ultrasound imaging using angular compounding”; 2004/0215075, entitled: “Ultrasonic elastography with angular compounding”; 2004/0210136, entitled: “Method and apparatus for imaging the cervix and uterine wall”, all naming the first inventor and hereby incorporated by reference.

Each of these strain measurements characterizes the flexibility of the plaque 45 under the pulsating force of flowing blood. The measurements are repeated for each pair of successive image data frames 42, 42′ and thus generate a set of time series waveforms 68, 70, and 72. Time series 68 provides variation in lateral strain 60, time series 74 provides variation in axial strain 64, and time series 72 provides variation in shear strain 66, each for a variety of points in the plaque 45.

Each of these time series 68, 70, and the 72 has a regular period tracking the cardiac cycle and thus the individual cardiac cycles may be “ensemble averaged” by aligning successive cardiac cycles with each other and performing a point by point averaging of corresponding points within each cardiac cycle. This statistical processing and/or other statistical processing techniques, indicated at process block 78 of FIG. 5, may provide a more robust measurement of these dynamic properties. Further in the maximum displacement, strain, or sheer during each cardiac cycle may be determined to provide the extracted parameter.

At process block 80, extracted parameters from the statistically processed time series 68, 70, and 72 may be applied to risk data, for example contained in a lookup table, to equate these extracted parameters to the risk presented to the patient by the plaque 45. Generally, for example, the peak-to-trough strain variation 75 may be determined for time series 68 for each of these measurements to provide three different views of the elasticity of the plaque 45. The present inventors have determined this extracted parameter of peak-to-trough strain variation 75 (maximum accumulated axial strain) applied to axial strain will range from 18% for softer plaques to 7% for calcified plaques.

The inventors have linked extracted parameters to the risk presented by the underlying plaque 45 from a study population of individuals who have been tested for cognitive decline and who have had ultrasonic measurements of the elasticity of their plaques 45. In the preferred embodiment, this study population was presented with a brief but reliable assessment of the domains of cognition including immediate memory (word list learning, paragraph recall), visuospatial ability (construct a complex figure, spatial orientation), language (confrontation naming, semantic fluency), attention (digit span forward, digit symbol substitution), delayed memory (word list, paragraph recall, complex figure), and a summary measure of overall cognitive performance (RBANS (Repeatable Battery for the Assessment of Neuropsychological Status) Total).

The inventors have determined that there is a significant correlation between peak-to-trough lateral strain variation 75 and the RBANS total performance (−0.79, p=0.02) with higher strain associated with poorer cognitive performance. In addition there are significant associations between these strain types and immediate memory (−0.793 p=0.03) and delayed memory (−0.88 p=0.009).

In the present invention cognitive studies of a standard population are used to generate a multidimensional risk function embodied, for example, in a lookup table being part of program 28 and relating one or more of these strain measurements to cognitive decline. This lookup table is used at process block 80 to provide a continuous range of risk values, for example, tracking the measured cognitive decline of the standard population. This risk value may be output on the display screen 18 for example as a number or in the form of a chart, for example a bar chart placing the risk within a range and optionally relating it to individual reference populations by age or the like. A threshold 90 with respect to this risk may be established to indicate when corrective procedures should be undertaken based on current and evolving standards of medical care.

This analysis of process block 80 may also make use of other data that may be obtained with respect to the plaque 45 and that may help improve the correlation including for example, scatterer size, and attenuation. Scatterer size indicates the diameter of acoustic scatterers and is particularly useful in an ultrasound imaging system and may be determined by an analysis of the spectra of the underlying radio frequency ultrasound data according to methods known in the art. Generally plaques 45 that are calcified and thus may provide more resistance to rupture tend to have smaller scatterer sizes (Faran: 120˜180 μm) whereas plaques 45 without calcification (softer plaques) have larger scatterer sizes (Faran: 280˜470 μm).

Similarly, attenuation as a function of frequency may be used to further characterize the plaques 45 with calcified regions showing increased attenuation with frequency (1.4˜2.5 db/cm/MHz) whereas plaques 45 having higher risk of rupture have lower attenuation 0.3˜1.4 db/cm/MHz).

It is specifically intended that the present invention not be limited to the embodiments and illustrations contained herein and the claims should be understood to include modified forms of those embodiments including portions of the embodiments and combinations of elements of different embodiments as come within the scope of the following claims.

While in the preferred embodiment, ultrasonic imaging is used for deducing strain, it will be understood to those of ordinary skill in the art that other imaging modalities may also be used including, for example, x-ray CT or MRI imaging. The use of these modalities for the measurement of tissue elastic properties is disclosed in U.S. Pat. No. 6,037,774, entitled: “Inertial driver device for MR elastography”; U.S. Pat. No. 6,862,468, entitled: “Systems and methods for magnetic resonance imaging elastography”, and U.S. Pat. No. 7,257,244 entitled: “Elastography imaging modalities for characterizing properties of tissue”; all in corporate it by reference. It will be further understood that although cognitive decline was used to establish the benchmark against which vulnerable plaques are classified, a similar classification could be derived from risk of vascular cognitive dementia, stroke or Alzheimer's disease with additional studies.

We claim:

1. An apparatus for a characterization of arterial plaque comprising:
an imaging system providing image data distinguishing plaque from at least a portion of a supporting arterial wall;
an electronic computer executing a stored program and receiving the image data to:
(1) isolate movement of the plaque from movement of the supporting arterial wall under a periodic force of pulsatile blood flow;
(2) analyze the movement of the plaque to characterize a risk of the plaque rupturing to produce harmful embolisms; and
(3) output an indication of the risk.

2. The apparatus of claim 1 wherein the analysis determines at least one of: axial strain in the plaque and axial displacement in the plaque.

3. The apparatus of claim 1 wherein the analysis determines at least one all of lateral strain of the plaque and lateral displacement of the plaque.

4. The apparatus of claim 1 wherein the analysis determines shearing strain in the plaque.

5. The apparatus of claim 1 wherein the imaging system is an ultrasonic imaging system.

6. The apparatus of claim 1 wherein the isolation of movement of the plaque determines movement of the portion of the supporting arterial wall and compensates this movement of the portion of the supporting arterial wall from movement of the plaque.

7. The apparatus of claim 1 wherein the isolation of movement of the plaque compares movement of the first portions of the plaque to movement of other portions of the plaque.

8. The apparatus of claim 1 wherein the electronic computer further provides a display of the image data and a cursor for identifying a region of plaque and a region of supporting arterial wall.

9. The apparatus of claim 1 further including a heartbeat monitoring system providing a timing reference to the electronic computer for processing the image data.

10. The apparatus of claim 1 wherein the computer program ensemble averages data used for the analysis for a plurality of cardiac cycles.

11. The apparatus of claim 1 wherein the computer program analyzes the movement over one cardiac cycle to compute a maximum deformation of the plaque to characterize the risk.

12. The apparatus of claim 1 wherein the analysis determines at least one all of: maximum accumulated axial strain in the plaque; maximum accumulated axial displacement in the plaque; maximum accumulated lateral strain of the plaque; and maximum accumulated lateral displacement of the plaque.

13. The apparatus of claim 1 wherein the imaging system is an ultrasound imaging system and the electronic computer further analyzes at least one of scatterer size and ultrasonic attenuation as a function of frequency in characterizing the risk of a plaque rupturing.

14. A method of characterizing of arterial plaque comprising:
(a) collecting a series of time images distinguishing plaque and at least a portion of a supporting arterial wall;
(b) using an electronic computer executing a stored program and receiving the images to:
(1) isolate movement of the plaque from movement of the supporting arterial wall under a periodic force of pulsatile blood flow;
(2) analyze the movement of the plaque to characterize a risk of the plaque rupturing to produce dangerous embolisms; and
(3) output an indication of the risk.

15. The method of claim 14 wherein the analysis determines at least one of axial strain in the plaque and axial displacement in the plaque.

16. The method of claim 14 wherein the analysis determines at least one of lateral strain in the plaque and a lateral displacement in the plaque.

17. The method of claim 14 wherein the analysis determines shearing strain in the plaque.

18. The method of claim 14 wherein the series of time images is collected using an ultrasonic imaging system.

19. The method of claim 14 wherein the isolation of movement of the plaque determines movement of portions of the plaque with respect to the portions of the supporting arterial wall.

20. The method of claim 14 wherein the isolation of movement of the plaque determines movement of portions of the plaque with respect to other portions of the plaque.

21. The method of claim 14 wherein the electronic computer further provides a display of the images and a cursor for identifying a region of plaque and a region of supporting arterial wall.

22. The method of claim 14 wherein the computer program further ensemble averages data derived from the images for a plurality of cardiac cycles in the analysis step.

23. The method of claim 14 wherein the series of time images is obtained with an ultrasound imaging system and the electronic computer further analyzes at least one of scatterer size and ultrasonic attenuation as a function of frequency in characterizing the risk of a plaque rupturing.

24. The method of claim 14 wherein the computer further characterizes the outputted risk by comparing the deformation of the plaque to deformations associated with at least one all of: a predetermined increased risk of cognitive decline, a predetermined increased risk of vascular cognitive dementia, a predetermined increased risk of stroke, a predetermined increased risk of Alzheimer's disease.

25. The method of claim 14 wherein the computer further characterizes the outputted risk by comparing the deformation of the plaque to deformations associated with a predetermined increased risk of vascular cognitive dementia.
